(19) United States
(12) Patent Application Publication
(10) Pub. No.: US 2025/0247597 A1
(43) Pub. Date: Jul. 31, 2025

(54) DEVICES, SYSTEMS, AND METHODS FOR IMAGING USING SHORTWAVE INFRARED LIGHT

(71) Applicants: THE BOARD OF TRUSTEES OF THE LELAND STANFORD JUNIOR UNIVERSITY, Stanford, CA (US); Helmholtz Zentrum Muenchen-Deutsches Forschungszentrum fur Gesundheit und Umwelt (GmbH), Neuherberg (DE)

(72) Inventors: Roy Park, Stanford, CA (US); Stella Yang, Stanford, CA (US); Tulio Valdez, Palo Alto, CA (US); Oliver Bruns, Neuherberg (DE); Thomas Stanley Bischof, Munchen (DE); Tjadina-Wencke Klein, Neuherberg (DE)

(21) Appl. No.: 19/182,519

(22) Filed: Apr. 17, 2025

Related U.S. Application Data
(63) Continuation of application No. PCT/US23/35447, filed on Oct. 18, 2023.
(60) Provisional application No. 63/417,288, filed on Oct. 18, 2022.
(60) Provisional application No. 63/429,081, filed on Nov. 30, 2022.

(51) Int. Cl. H04N 23/50; G02B 23/24; H04N 23/55; H04N 23/56; H04N 23/63

(52) U.S. Cl. CPC H04N 23/555; H04N 23/55; H04N 23/56; H04N 23/63; G02B 23/243; G02B 23/2469

(57) ABSTRACT

Imaging devices, systems, and methods are provided for imaging using narrow bands of short wavelength infrared (SWIR) light, e.g., to image at water absorption wavelength and/or to image through blood. In one example, an imaging device is provided for imaging during a medical procedure that includes a proximal end, a distal end sized for introduction into a subject's body, and an imaging element carried by the distal end, a light source coupled to the imaging device to deliver one or both of narrow band short wavelength infrared (SWIR) light and visible light, and a camera for acquiring images via the imaging element.

### RELATED APPLICATION DATA

The present application is a continuation of co-pending International Application No. PCT/US2023/035447, filed Oct. 18, 2023, which claims benefit of U.S. provisional applications Ser. Nos. 63/417,288, filed Oct. 18, 2022, and 63/429,081, filed Nov. 30, 2022, the entire disclosures of which are expressly incorporated by reference herein.

### STATEMENT REGARDING FEDERALLY SPONSORED RESEARCH AND DEVELOPMENT

None.

### TECHNICAL FIELD

The present application relates to medical devices and, more particularly to devices, systems, and methods for imaging during surgical or other medical procedures, e.g., using shortwave infrared (SWIR) light.

### BACKGROUND

Imaging devices are known that may be introduced into a patient's body during a surgical procedure, e.g., to locate and/or identify anatomical structures during navigation and/or to identify target locations for diagnosis and/or treatment. However, within body lumens, e.g., blood vessels and/or chambers of the heart, blood may obscure visualization using visible light.

In addition, the diagnosis of cerebrospinal fluid (CSF) leaks can be challenging,

and a missed or delayed diagnosis can lead to potentially life-threatening complications such as meningitis. With recent advancements in endoscopic skull base surgery, the incidence of CSF leaks continues to rise, with as high as 30.1% of cases having an intraoperative CSF leak, making iatrogenic causes the leading etiology. In this setting, and particularly in the acute postoperative period, CSF rhinorrhea can be difficult to distinguish clinically from blood and mucus drainage. Other scenarios where a CSF leak may be difficult to discern and locate are in the presence of craniofacial trauma and spontaneous CSF leaks.

Existing options for CSF leak diagnosis such as intrathecal fluorescein or Beta2 transferrin have limitations in immediacy of results and introduce potential morbidity. For example, identifying CSF rhinorrhea currently requires invasive procedures such as intrathecal fluorescein which requires a lumbar drain placement. Fluorescein is also known to have rare but significant side effects including seizures and death. As the number of endonasal skull base cases increase, the number of CSF leaks have also increased for which an alternative diagnostic method would be highly advantageous to patients.

Accordingly, devices and methods that facilitate imaging during medical procedures, e.g., to identify CSF and/or to image through blood, would be useful.

### SUMMARY

The present application is directed to medical devices and, more particularly to devices, systems, and methods for imaging during surgical or other medical procedures, e.g., using shortwave infrared (SWIR) light.

For example, the devices, systems, and methods herein may facilitate identifying CSF leaks based on water absorption in the SWIR, e.g., without need for intrathecal contrast agents. In one example, the devices herein may be adapted to the anatomy of the human nasal cavity while maintaining low weight and ergonomic characteristics of current surgical instruments.

The devices may also enhance imaging in other procedures and/or contexts, e.g., to image at wavelengths within water absorption bands of light, e.g., to facilitate distinguishing different tissues and/or fluids, e.g., to distinguish fat tissue, which substantially reflects light at water absorption wavelengths, from other tissues that contain substantial amounts of water and, consequently, absorb light at water absorption wavelengths. For example, the imaging devices herein may facilitate identifying lymph nodes or other anatomical structures within a field of view of the imaging devices, or may facilitate identifying fat content in organs, such as the liver, or other tissues.

In addition, the imaging devices and methods herein may be used during other surgical or medical procedures. For example, the imaging devices may be configured to be introduced into one or more body lumens, e.g., a patient's vasculature during an interventional procedure. In addition, the imaging devices herein may be used outside of a subject's body, e.g., mounted on an arm or other structure that may otherwise be positioned adjacent a patient's body during an open procedure. For example, the imaging devices herein may be mounted on a movable arm fixed to, above, or otherwise adjacent a surgical table such that a surgeon may manipulate the imaging device to generate images during a procedure. The imaging devices herein may also be used in a pathology suite, e.g., to facilitate analyzing tissue samples and the like.

As used herein, “shortwave infrared” light or SWIR refers to the electromagnetic spectrum ranging from about one thousand to two thousand nanometers (1000-2000 nm), which is not visible to the human eye and conventional silicon-based cameras. Compared to traditional visible and near-infrared (NIR) fluorescence imaging, SWIR is capable of providing greater contrast, sensitivity, and penetration depths. Moreover, tissue components such as water, lipids, and collagen have more prominent SWIR absorption features than corresponding features seen in the visible and near-infrared. Fluid in the middle ear, for example, shows strong light absorption between 1,400 and 1,550 nm. As a result, straightforward middle ear fluid detection in a model using a SWIR otoscope is possible and may be used to diagnose otitis media with greater accuracy than is currently possible with a standard otoscope.

Also used herein, “narrow band” light refers to light produced in a relatively small region of wavelengths of light, e.g., centered on a peak or center wavelength, in contrast to broad band light, such as visible light. For example, a light source such as a laser may provide a narrow band of light centered at a desired wavelength and a narrow region on either side of the desired wavelength, e.g., not more than about +/−five nanometers, +/−two nanometers, or +/−one nanometer. For other light sources, long pass (LP) or band pass (BP) filters may be used to provide a desired narrow band of light for imaging, as described further elsewhere herein.

In accordance with one example, an imaging device is provided that includes an imaging element, a light source configured to generate short wavelength infrared (SWIR) light to the imaging element at a narrow band centered at one or more water absorption bands, such as about 1450 nm, 1480 nm, 1485 nm, or 2000 nm, and a camera for acquiring images via the imaging element. A display may be coupled to the camera to display images acquired by the camera, e.g., to facilitate imaging during a surgical or other medical procedure.

In accordance with another example, an imaging device is provided that includes an elongate member comprising a proximal end, a distal end, and an imaging element carried by the distal end; a light source coupled to the proximal end configured to generate short wavelength infrared (SWIR) light to the imaging element at a narrow band centered at one or more water absorption bands, such as about 1450 nm, 1480 nm, 1485 nm, or 2000 nm; and a camera for acquiring images via the imaging element.

In accordance with yet another example, an imaging device is provided that includes an elongate member comprising a proximal end, a distal end sized for introduction into a subject's body, and an imaging element carried by the distal end; a light source coupled to the proximal end to selectively deliver one or both of short wavelength infrared (SWIR) light and visible light source to facilitate navigation of the distal end within the body; and a camera for acquiring images via the imaging element. In one example, the light source is configured to generate SWIR light to the imaging element at a narrow band centered on a desired wavelength, e.g., at one or more water absorption bands, such as about 1450 nm, 1480 nm, 1485 nm, or 2000 nm. In another example, the light source is configured to generate SWIR light to the imaging element at one or more wavelengths to allow imaging through blood, e.g., a narrow band of SWIR light at about 1300 nm, 1450 nm, 1550 nm, or 1650 nm. Optionally, the imaging device is coupled to a display that may display images from the camera, e.g., to selectively display SWIR light images, visible light images, or both SWIR and visible light images superimposed on one another on the display.

In accordance with another example, a method is provided for imaging, e.g., during a medical procedure that includes generating short wavelength infrared (SWIR) light at a narrow band centered at one or more water absorption bands, such as about 1450 nm, 1480 nm, 1485 nm, or 2000 nm, and acquiring images via the imaging element. The images may be displayed, e.g., to facilitate imaging during a surgical or other medical procedure.

In accordance with still another example, a method is provided for imaging within a passage of a subject's body that includes introducing an imaging device into a target location within the body; delivering visible light via the imaging device into the passage; acquiring images via the imaging device within the passage. When an imaging field of the imaging device is obscured by blood, the method further comprises delivering shortwave infrared light via the imaging device into the passage; and acquiring images via the imaging device within the passage to image through the blood.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 is a schematic of an example an imaging system including an endoscope, a light source, and a camera, e.g., configured to deliver one or both of visible light and shortwave infrared light to facilitate imaging through various bodily fluids.

FIG. 2 is a side view of an exemplary endoscope and camera that may be included in the imaging system of FIG. 1.

FIGS. 2A and 2B are details of a distal end of the endoscope of the imaging device of FIG. 2.

FIGS. 3A-3K show various images taken using illumination in the shortwave infrared range.

FIGS. 4A and 4B are side and end views, respectively, of another example of an imaging device.

FIGS. 5A-5C are exemplary images acquired using the imaging device of FIGS. 4A and 4B.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

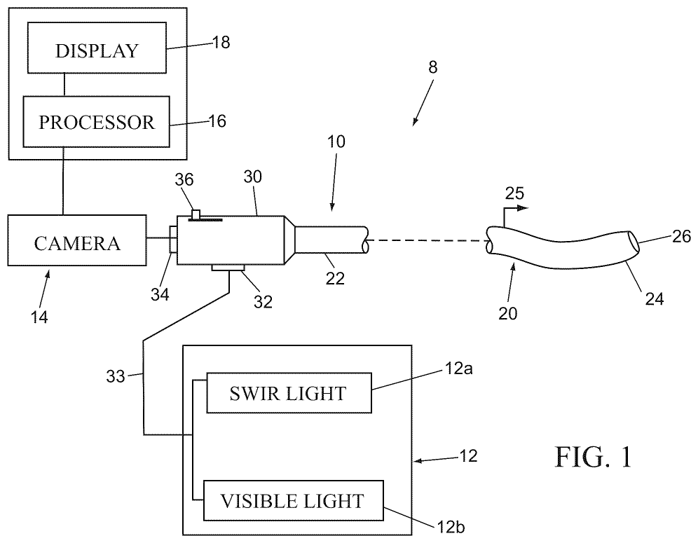

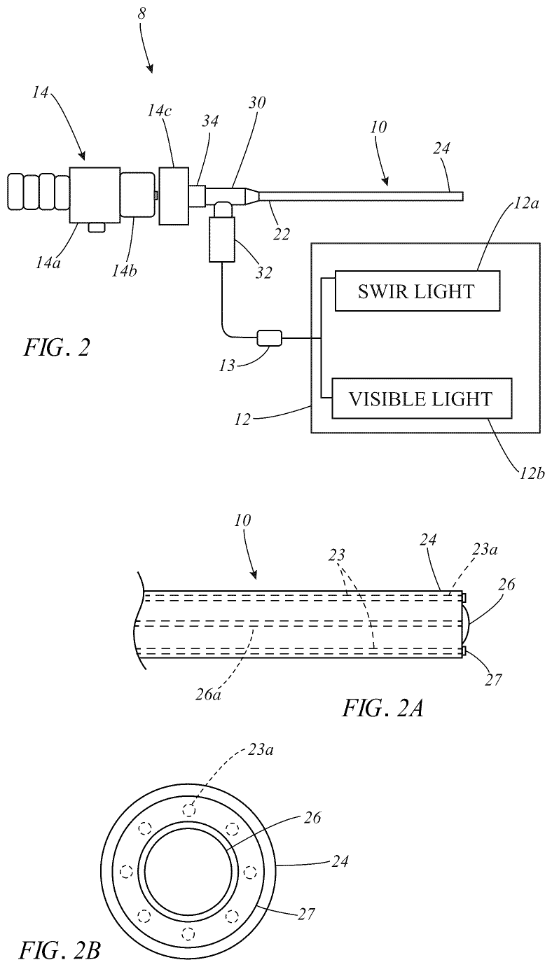

Turning to the drawings, FIGS. 1 and 2 show an example of a device or system 8 for imaging during a medical procedure, e.g., during a diagnostic and/or interventional procedure, or during a surgical procedure in which one or more instruments are introduced into one or more body lumens, passages, and/or regions within a patient's body. As shown in FIG. 1, the system 8 includes an imaging device, e.g., an endoscope, boroscope, or other elongate member 10, a light source 12, and a camera 14. Optionally, the system 8 may include one or more additional components, e.g., a processor 16 coupled to the camera 14 for processing images from the camera 14 and/or a display 18 coupled to the processor 16 (and/or camera 14 directly) for presenting images to the user, as described further elsewhere herein. Although the system 8 is shown including an endoscope 10, it will be appreciated that other imaging devices capable of being introduced into a patient's body may be provided including the light source 12, camera, 14, and/or other components of the examples described herein.

Generally, the endoscope 10 includes a shaft or elongate member 20 including a proximal end 22 including a handle or hub 30, a distal end 24 sized for introduction into a patient's body, and an imaging element 26 carried by the distal end 24. The elongate member 20 may include one or more lumens (not shown) extending between the proximal and distal ends 22, 24. For example, one or more fiber lumens may be provided within which one or more optical fibers, e.g., a multiple fiber bundle or individual fibers, may be received that are coupled to the imaging element 26 for delivering light and/or acquiring images beyond the distal end 24, as described further elsewhere herein. Alternatively, one or more optical fibers may be embedded within a wall of the elongate member 20, such that fiber(s) extend between the proximal and distal ends 22, 24. Optionally, an infusion and/or instrument lumen may be provided that extends from a port (not shown) on the hub 30 to an outlet in the distal end 24 (not shown), e.g., to deliver fluids beyond the distal end 24 and/or introducing one or more instruments through the endoscope 10 during a procedure.

The elongate member 20 may be substantially flexible, semi-rigid, and/or rigid along its length, and may be formed from a variety of materials, including plastic, metal, and/or composite materials, as is well known to those skilled in the art. For example, as shown in FIG. 1, the elongate member 20 may be substantially flexible along a distal portion 25 terminating at the distal end 24 to facilitate advancement through tortuous anatomy, and/or may be semi-rigid or rigid adjacent the proximal end 12 to enhance pushability and/or torqueability of the endoscope 10 without substantial risk of buckling or kinking.

Optionally, for a steerable distal portion 25, the endoscope 10 may include one or more wires or other steering elements slidably received within respective steering lumen(s) (not shown) extending from the proximal end 22 to a fixed location within or at the distal end 24 to allow the distal portion 25 to be bent or otherwise steered, e.g., to allow the distal end 14 to be introduced into a patient's body, e.g., through the patient's vasculature into a chamber of the heart or other location in which blood may potentially obscure the imaging field, to perform a surgical or other medical procedure. In this example, one or more actuators, e.g., a slider or rotating dial (not shown), may be provided on the hub 30 that is coupled to the steering element(s) to manipulate the shape and/or curvature of the distal end 14 during introduction. Alternatively, the distal portion 25 may be sufficiently flexible that the distal portion 25 may be advanced over a guidewire or other rail previously introduced into the patient's body, e.g., via an instrument lumen of the endoscope 10 (not shown).

Alternatively, as shown in FIG. 2, the elongate member 20 may be straight and substantially rigid between the proximal and distal ends 22, 24, e.g., to facilitate introduction and/or manipulation of the distal end 24 from the proximal end 22, for example, to facilitate introduction of the distal end 24 into a patient's sinus, ear canal, and/or other body orifice while holding the proximal end 22.

With additional reference to FIGS. 2A and 2B, the imaging element 26 may include one or more lenses, filters, and/or other components optically coupled to a fiberoptic cable, liquid light guide, and the like 26a extending proximally from the distal end 24. For example, as shown, a lens 26 may be provided on the distal end 24 that receives reflected light within a field of view beyond the distal end 24 with the light signals delivered to the camera 14 via the cable 26a, as described further elsewhere herein.

In addition, one or more optical fibers may be provided that transmit light distally beyond the distal end 24. For example, as shown in FIG. 2A, a plurality of optical fibers 23 may be embedded in a wall of the endoscope 10 that extend to the distal end 24, e.g., with distal ends 23a of the fibers 23 arranged around the imaging element 26 as shown in FIG. 2B, to transmit light distally from the distal end 24. For example, the fibers 23 may selectively transmit SWIR and/or visible light from the light source 12, e.g., to illuminate tissues, fluids, and/or other structures within the field of view of the imaging element 26, and the imaging element 26 may receive reflected light that is delivered to the camera 14, e.g., for processing and/or presentation on the display 18, as described further elsewhere herein.

Optionally, one or more filters, polarizers and the like may be provided on the distal end 24. For example, as shown in FIGS. 2A and 2B, a polarizer 27 may be provided on the distal end 24 that covers the ends 23a of the light fibers 23, e.g., an annular polarizer sheet permanently or removably attached to the distal end over the ends 23a, to polarize incident light transmitted from the distal end 24. As described elsewhere herein, such a polarizer, e.g., in cooperation with a polarizer on the camera 14, may reduce undesired reflections in images being received by the camera 14.

As best seen in FIG. 2A, the distal end 24 may have a substantially planar surface, e.g., including the imaging element 26 and polarizer 27. Alternatively, the distal end 24 may have a tapered and/or rounded shape, if desired (not shown).

Alternatively, a camera, e.g., a CMOS, CCD, and the like, may be carried on the distal end 24 and one or more wires or cables (not shown) may extend proximally from the camera to the hub 30 that may be connected to the processor 16 and/or display 18 via an electrical connector on the hub 30, e.g., for coupling the device 10 to a power source, such as a battery or controller (not shown). Alternatively, a battery or other power source may be carried within the hub 30. In addition or alternatively, one or more of the light sources, e.g., LEDs, and the like (not shown) may be carried on the distal end 24 and one or more wires or cables may extend proximally from the distal end 24, e.g., to a power source (not shown) connected to the hub 30, to deliver power and/or otherwise activate/deactivate the light source(s).

Returning to FIGS. 1 and 2, the hub 30 may include one or more connectors, e.g., for coupling the light source 12 and/or camera 14 to the endoscope 10. For example, a first connector 32 may be provided on the hub 30 configured to connect to a fiberoptic cable, liquid light guide, or other cable 33 to optically couple the light source 12 to the optical fiber(s) 23 within the endoscope 20, thereby optically coupling the light source 12 to the endoscope 10 to deliver light beyond the distal end 24. In addition, a second connector 34, e.g., a C-mount, video coupler, and the like, may be provided on the hub 30 configured to optically couple the camera 14 to the imaging element 26, e.g., via the cable 26a. One or more optical couplings (not shown) may also be provided within the hub 30, e.g., to direct light from the light source 12 to the fiber(s) 23 to the distal end 24 of the endoscope 10 and/or to direct light from the imaging element 26 to the camera 14.

With continued reference to FIGS. 1 and 2, the light source 12 may include a light box separate from the endoscope 10, including one or more LEDs, laser, or other light sources configured to deliver one or more desired wavelengths of light to the endoscope 10, e.g., to transmit from the distal end 24 via the fibers 23. Alternatively, one or more light sources may be provided within the endoscope (not shown), if desired.

For example, as shown, the light source 12 may include one or more SWIR light sources 12a, e.g., lasers, LEDs, Halogen lights, and the like, configured to generate SWIR light, e.g., between about 1200-2500 nm, or between about 1200-1550 nm. In various examples, the SWIR light source 12a may be configured to transmit a narrow band of light within the SWIR range, e.g., at wavelengths centered at about 1300 nm, 1450 nm, 1480 nm, 1485 nm, 1550 nm, 1650 nm, or 2000 nm.

For example, the SWIR light source 12a may include a laser configured to generate a narrow band of SWIR light centered at a desired wavelength, e.g., centered at one or more water absorption bands, such as about 1450 nm, 1480 nm, 1485 nm, or 2000 nm. For this application, a laser light source, such as model L1480G1 from Thorlabs, may be provided that generates a narrow band at about 1480 nm or 1485 nm +/−5 nm (or narrower) without requiring filters, which may enhance contrast in images acquired by the camera.

Alternatively, one or more filters 13 may be provided to narrow and/or otherwise filter the SWIR light, e.g., if the light source 12a transmits broadband SWIR light. For example, with LEDs or Halogen lights, it may be desirable to provide one or more filters to narrow the band of wavelengths delivered from the distal end 24. For example, one or more long pass or band pass filters 13 may be optically coupled between the light source 12 and the endoscope 10 to narrow the band of light delivered from the distal end 24. Examples of SWIR devices that may be included in the devices and systems herein are disclosed in U.S. Pat. No. 9,986,915, the entire disclosure of which is expressly incorporated by reference herein.

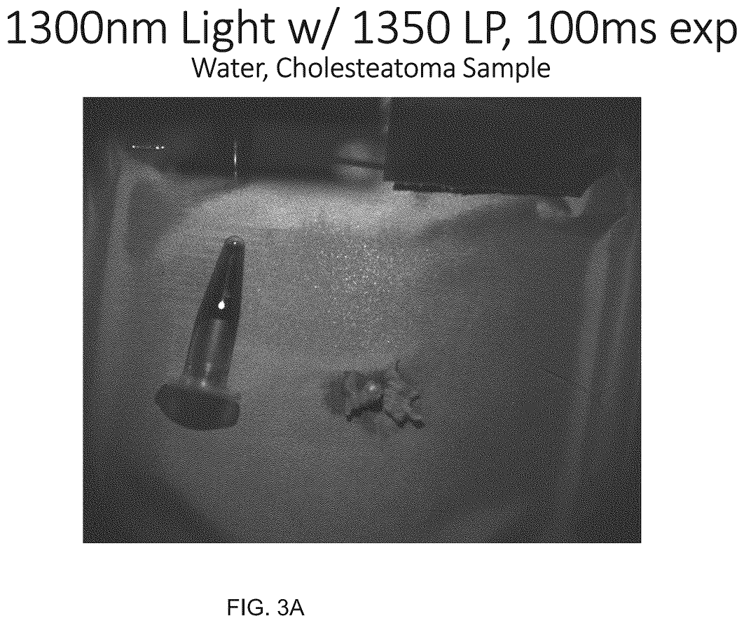

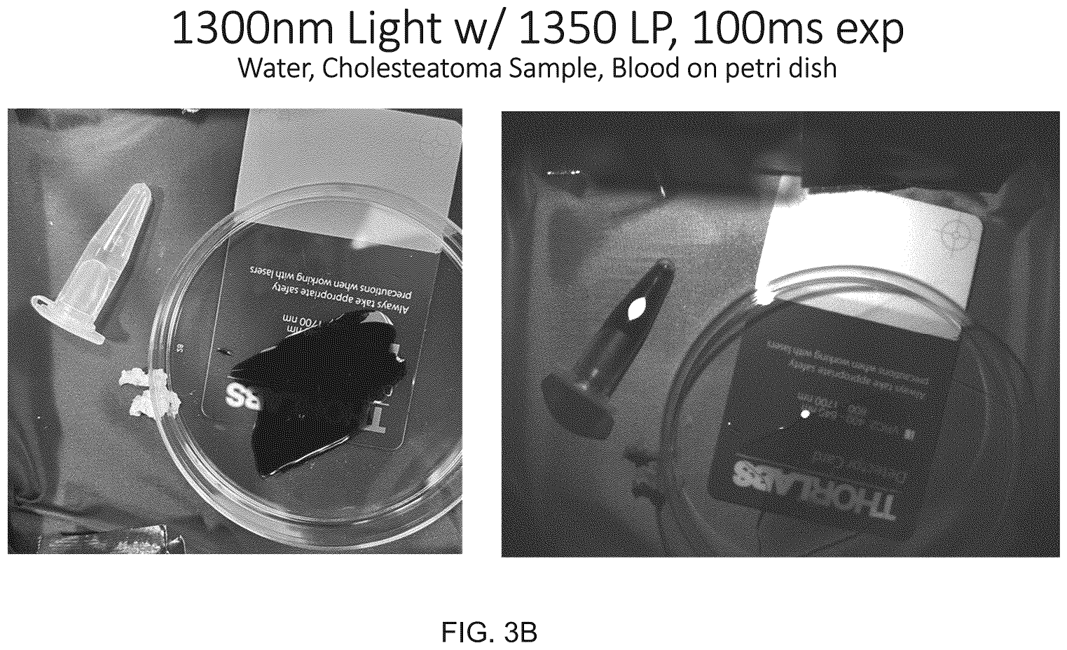

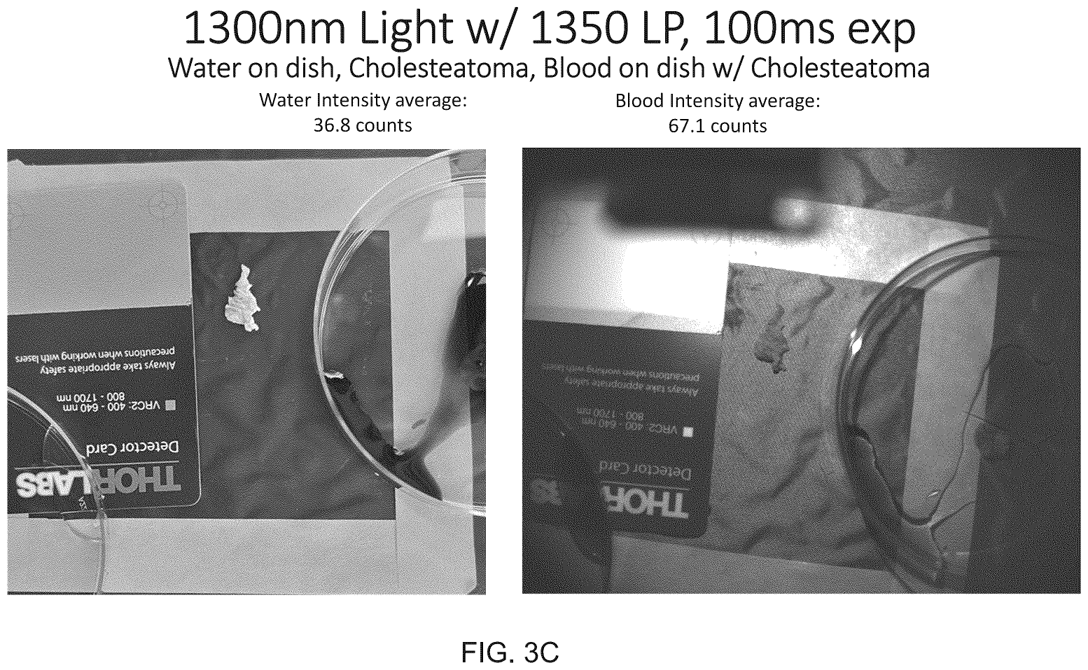

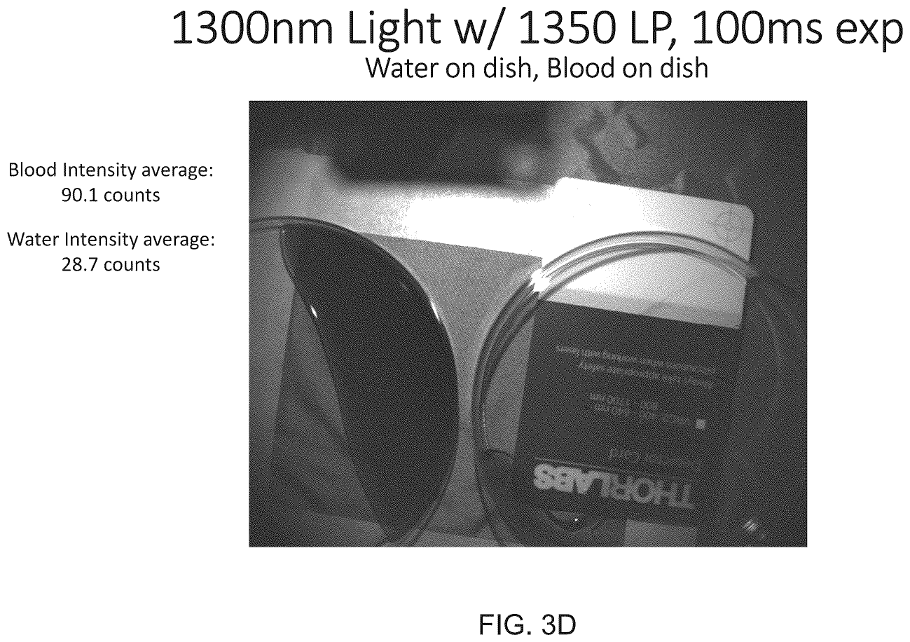

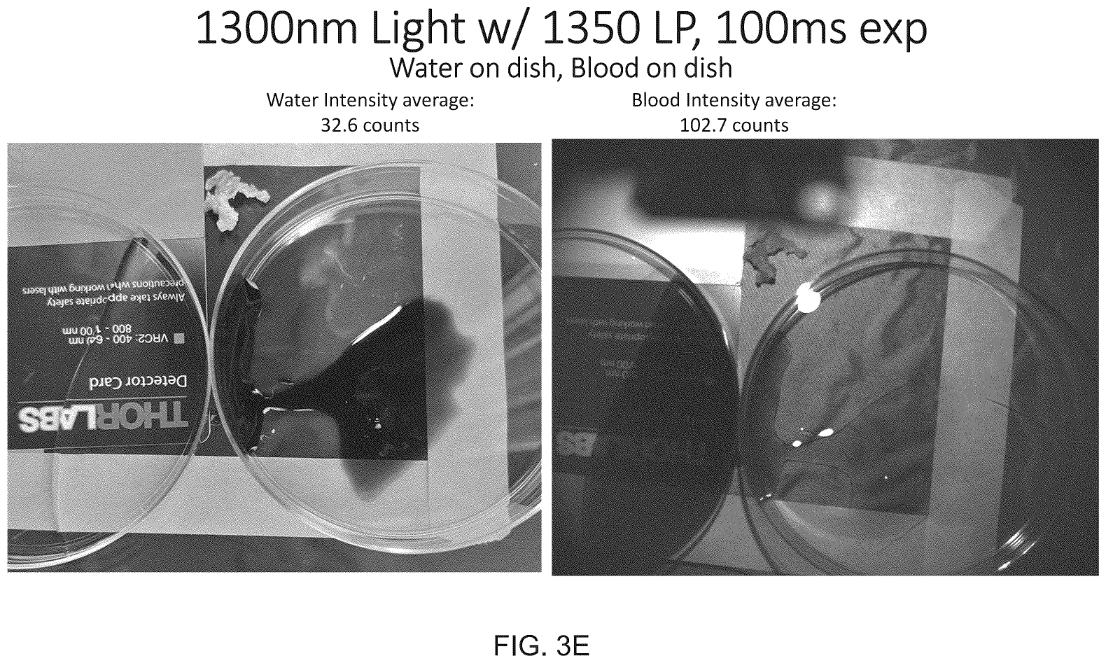

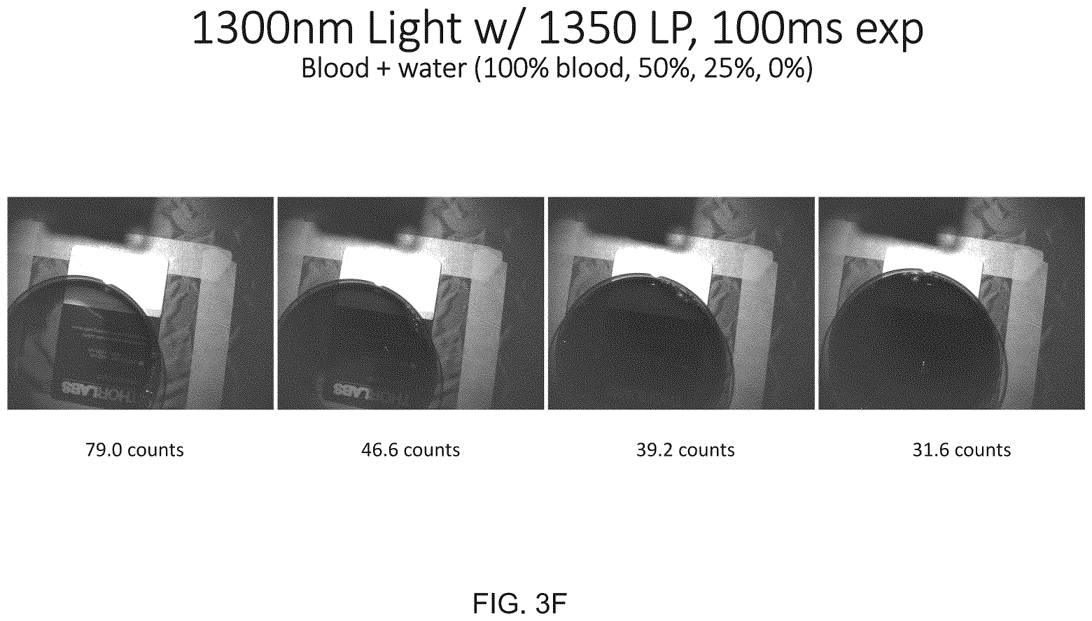

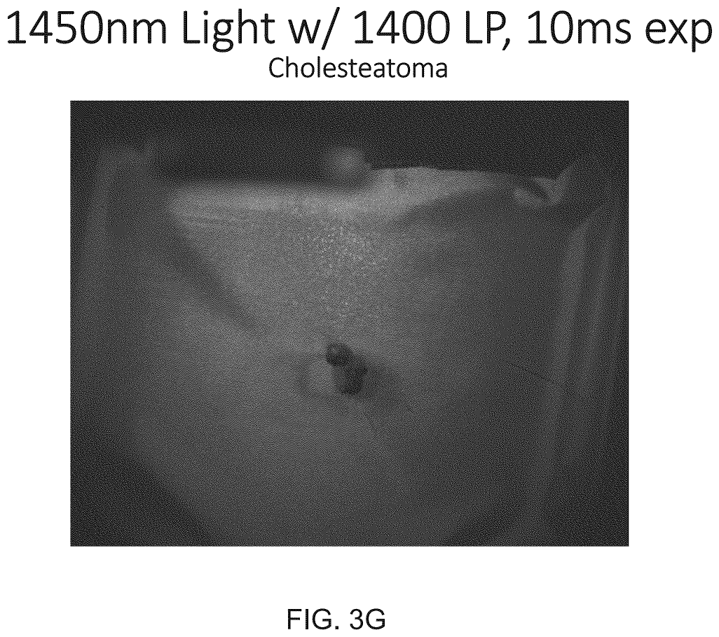

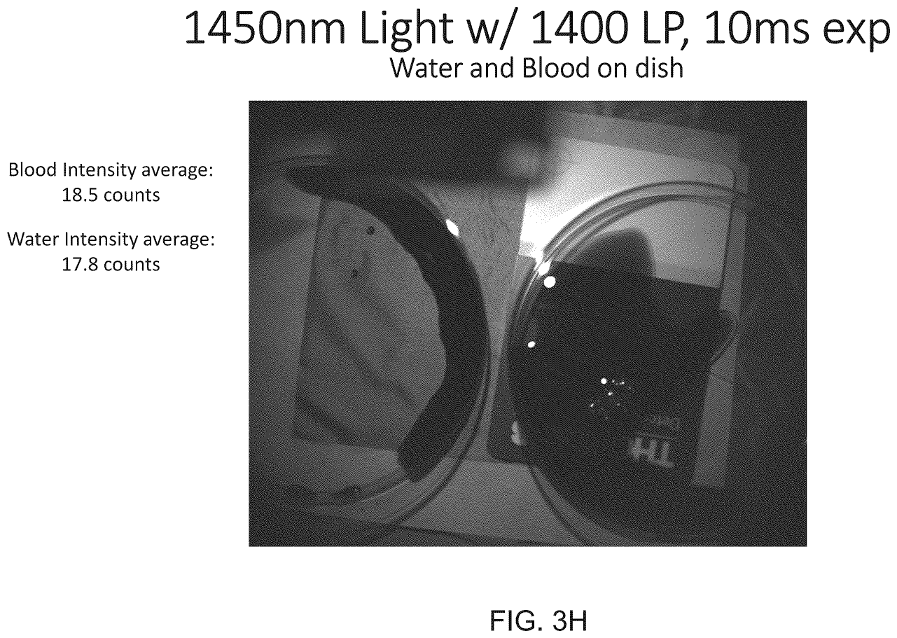

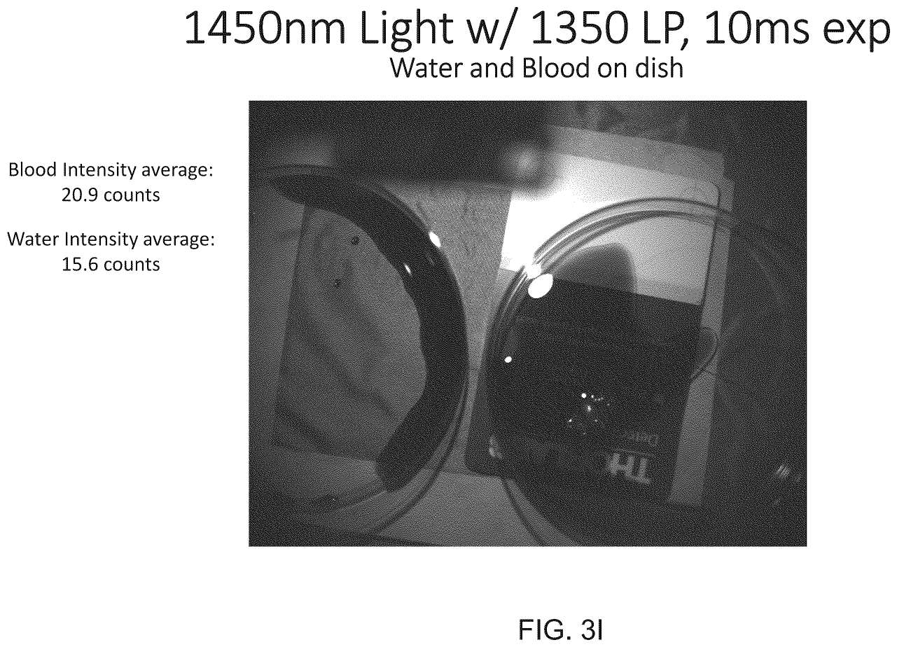

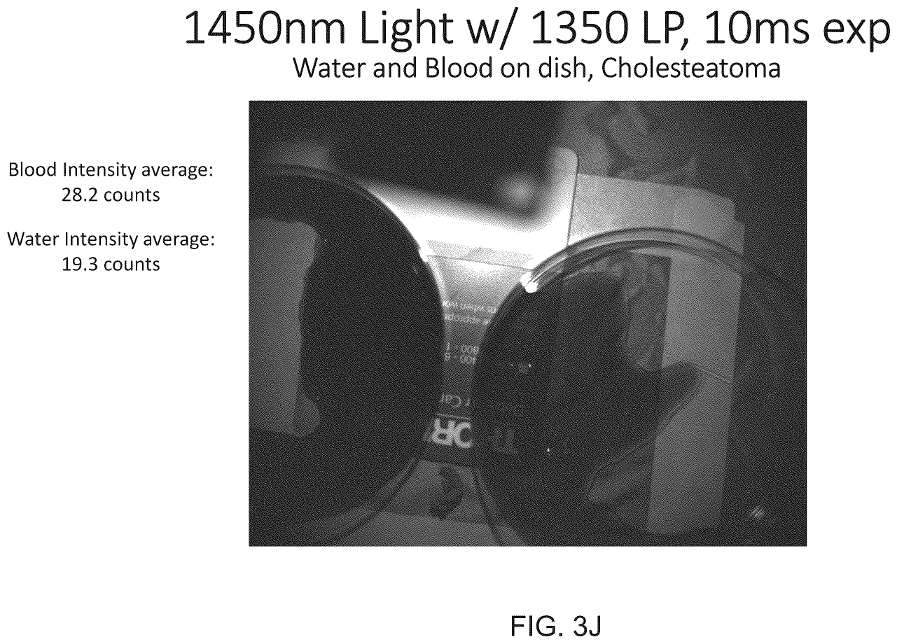

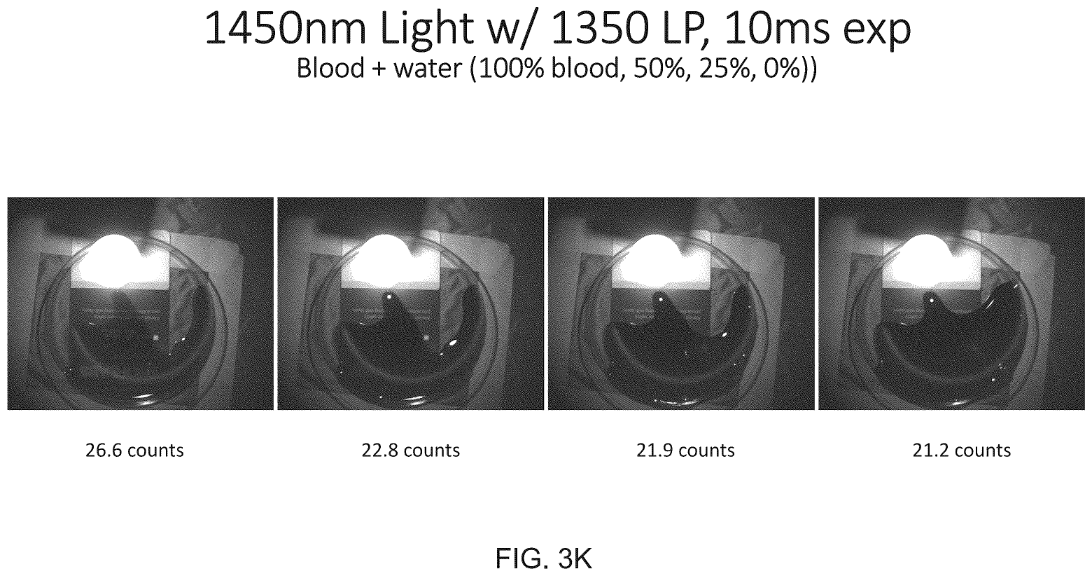

In addition or alternatively, the SWIR light source 12a may be configured to generate light at wavelengths at which blood may be substantially transparent, e.g., a narrow band of SWIR light at about 1300 nm, 1450 nm, 1550 nm, or 1650 nm. A light source, such as a laser, may be capable of generating a desired narrow band without filters or, one or more filters may be provided, as desired. For example, as shown in FIGS. 3A-3F, a 1350 nm long pass filter was used to filter light from a 1300 nm LED light source, i.e., to block all light below 1350 nm, while, as shown in FIGS. 3G-3K, a 1400 nm or 1350 nm LP filter was used along with a 1450 nm LED, to enhance contrast in the images acquired.

In addition, the light source 12 also includes an LED or other visible light source 12b, e.g., configured to deliver broadband white light to allow for surgical navigation when manipulating the endoscope 10 within the subject's body and/or other conventional imaging. Optionally, the light source 12 may also include one or more near IR LEDs or laser sources (not shown) configured to deliver near IR light, e.g., between about 600-825 nm or between about 785-808 nm, to allow excitation of ICG or other fluorescent dye administered to the subject, e.g., as disclosed in U.S. Publication No. 2022/0087592, the entire disclosure of which is expressly incorporated by reference herein.

The hub 30 may include one or more actuators, e.g., switch 36, that may be actuated to turn desired light sources off and on. For example, a single switch 36 may be provided on the hub 30 that may be moved between a first/off position where the light source 12 is completely off or isolated from the fiber(s) within the elongate member 20, a second position where the SWIR light source 12a is activated (without activating the visible light source 12b), and a third position where the visible light source 12b is activated (with the SWIR light source 12a remaining on or turned off). For example, the switch 36 may be used to alternate between activating the SWIR light source 12a and the visible light source 12b. Alternatively, separate switches or other actuators may be provided to selectively activate the light sources 12a, 12b, e.g., on the hub 30, on a floor step-on switch (not shown) coupled to the hub 30, on the light source 12 itself, and the like.

The camera 14 may include a detector 14a, e.g., a CMOS, CCD, InGaAs, or other sensor, configured to acquire images within the SWIR bandwidth that may be coupled to the hub 30 via the second connector 34, e.g., a C-mount, to acquire images from the imaging element 26 on the distal end 24 of the endoscope 10. Optionally, the camera 14 may include one or more filters 14b, or separate filters may be coupled between the detector 14a and the second connector 34, e.g., to limit the SWIR light received by the camera 14 to a desired narrow band, and the like. For example, the endoscope 10 may include a C-mount adapter on the proximal end that includes SWIR optics that are configured to allow a user to change filters, e.g., within the SWIR region. In addition or alternatively, one or more filters may be provided at the tip of the endoscope, e.g., part of the imaging element 26.

Optionally, one or more polarizers may be provided between the detector 14a and the imaging element 26. For example, a video coupler 14c may be provided that includes a lens and a polarizer (not shown), that may be optically coupled to the detector 14a to polarize light from the imaging element 26. For example, if the SWIR light source 12a is a laser, the coherent light may generate undesired reflections in images delivered to the camera 14. To minimize such reflections, a light polarizer 17, e.g., on the distal end over the ends 23a of the light fibers 23, and a camera polarizer, e.g., in the video coupler 14c may be oriented orthogonally relative to one another. Optionally, the video coupler 14c may be rotatable around a longitudinal axis of the endoscope 10, if desired, e.g., to adjust the degree of polarization.

The camera 14 may include a single detector 14a capable of processing both SWIR and visible reflected light from the imaging element 26. Alternatively, a separate detector may be provided for receiving each of the SWIR and visible light, e.g., which may be activated only when the corresponding light source is activated or otherwise coupled to deliver light from the distal end 24 of the endoscope 10. Alternatively, reflected light captured by the imaging element 26 may be split within the hub 30 or camera 14 to deliver light to the detectors.

With continued reference to FIG. 1, the camera 14 may be connected to a processor 16 and/or display 18, e.g., within a control box, such that signals from the camera 14 may be processed by the processor 16 for presentation on the display 18. Optionally, if desired, the light source 12 may also be provided within the control box to provide a single component to which the endoscope 10 may be connected to use the system 8. Alternatively, the camera 14 may also be provided within the control box. For example, in this alternative, a single connector may be provided on the hub 30 of the endoscope 10 that be connected to a corresponding connector on the control box to allow light to be delivered to the imaging element 26 from the light source(s) within the control box and to deliver image signals to be conveyed from the imaging element 26 to the camera within the control box (or electrical signals from a camera carried on the distal end 24 to the processor).

The system 8 may be used during a medical procedure to acquire images of desired tissues, fluids, and/or other structures within a patient's body. For example, in one method, a rigid endoscope 10, such as that shown in FIG. 2, may be introduced into a patient's sinus cavity, ear canal, or other body orifice to detect CSF leaks. In this example, the SWIR light source 12a may be configured to generate light at narrow band centered at one or more water absorption bands, such as about 1450 nm, 1480 nm, 1485 nm, or 2000 nm. The visible light source 12b may be activated at any time to generate visible images that may be presented on the display 18, e.g., to facilitate navigation into the orifice. When desired the SWIR light source 12a may be activated to generate images to facilitate identifying whether CSF is present. The visible images presented on the display 18 may be replaced with SWIR images or, alternatively, a composite image may be generated by the processor 16 for presentation on the display 18 including the SWIR images superimposed on the visible light images.

Given that CSF has been found to have absorption characteristics similar to water, SWIR light transmitted within narrow water absorption bands will be absorbed by CSF, which will present as dark regions on the SWIR images, as opposed to other tissue or fluids that may be present, that may readily reflect the SWIR light and appear bright in the images. Thus, a surgeon or other medical personnel may use the imaging devices herein to facilitate identifying CSF leaks without requiring fluorescent dyes or other contrast agents.

Further, using narrow band SWIR light within water absorption bands may provide additional advantages over other imaging modalities. For example, the imaging devices herein may be used in open surgical procedures to acquire SWIR images of a patient's body. Rather than using an endoscope to acquire images, an imaging device may be mounted to an arm or other structure adjacent a patient, e.g., to a surgical table, overhead structure, and the like within a surgical environment. The imaging device may include a SWIR light source, e.g., configured to generate a narrow band within a water absorption band, and a camera configured to acquire SWIR light images, which may be presented on a display. Thus, a surgeon may manipulate the imaging device to orient the camera towards target structures within a patient's body during a procedure and acquire SWIR images to guide the procedure.

One advantage of these imaging devices is that ambient sunlight will not affect images acquired with SWIR light within water absorption bands since the sunlight passing through the atmosphere of the earth naturally filters light at higher wavelengths, e.g., within the infrared spectrum range that includes the water absorption bands. Thus, the imaging devices herein may be used in locations that do not provide darkened conditions, such as an operating room that allows ambient light in, pathology suites, and the like. For example, SWIR images may be used to identify and/or analyze a patient's tissues in real time during a surgical procedure or tissue sample, e.g., identify lymph nodes or other organs within a patient's body or in tissue samples, to identify fat content in an organ such as the liver or other tissues, and the like.

In another method, the imaging devices herein may be used during a surgical procedure in which the endoscope 10 is introduced into a patient's body, e.g., into the patient's vasculature or other body passage in which dry or old blood may potentially obscure imaging using visible light. For example, initially, the visible light source 12b may be activated and images acquired to facilitate manipulation of the distal end 24 into a patient's body. Thus, visible images presented on the display 18 may facilitate introduction of the distal end 24, e.g., while using one or more steering elements in the endoscope 10 to navigate the distal end 24 and/or advancing the distal end 24 over a guidewire (not shown) to the target location.

At any time, if the field of view becomes obscured or the operator suspects blood may be present, the SWIR light source 12a may be activated (e.g., turning off or leaving on the visible light source), e.g., using the one or more actuators 36 on the hub 30, LED box 12, or elsewhere for selectively activating and deactivating the light sources. The SWIR images may allow the operator to image through any blood to facilitate further manipulation of the distal end 24 and/or performing a procedure at the target location. Optionally, the SWIR images may be provided separate from the visible images or they may be superimposed into a single composite set of images to facilitate the operator performing the procedure.

FIGS. 3A-3K show exemplary images demonstrating use of the imaging devices herein. For example, images acquired using visible light illumination may allow identification of tissue structures as long as blood is not present. However, as the amount of blood increases, visible light may become ineffective due to the opacity of blood to visible light. Consequently, if the images become obscured, the operator may activate the SWIR light source, at a wavelength at which blood is substantially transparent, to allow imaging to continue during the procedure. At any time, if the operator believes there is not a large amount of blood present, the user may activate the visible light source 12b (and optionally turn off the SWIR light source 12a), e.g., to facilitate directing the distal end 24 of the endoscope 10 within the patient's body and/or to observe one or more instruments introduced via the endoscope 10 to perform a surgical or other procedure. The resulting system and method may provide an effective method for imaging regardless of the fluid present within the body passage within which the device is introduced.

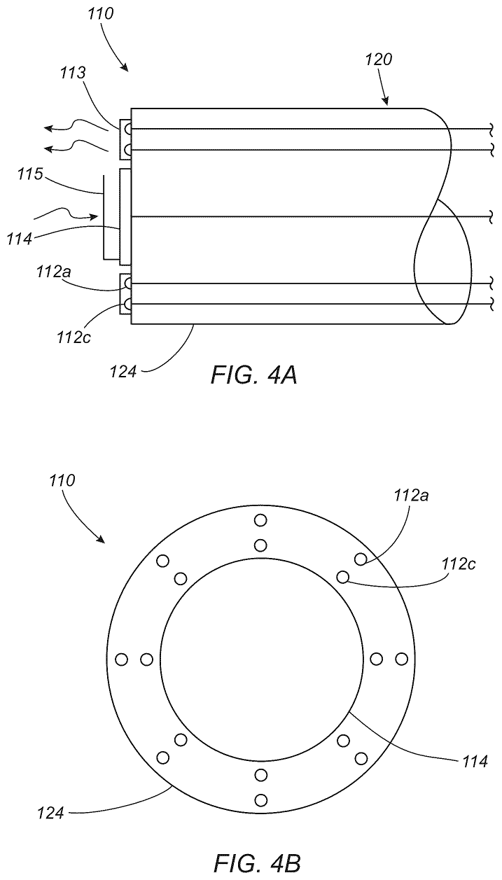

Turning to FIGS. 4A and 4B, a distal end 124 of another example of an imaging device 110 is shown, which may be generally constructed similar to the other imaging devices described wherein, e.g., including a flexible, semi-rigid, or rigid shaft 120 terminating in a distal end 124 that includes an imaging element 114 and a plurality of light sources 112. As shown, the light sources 112 may include first and second arrays of light sources 112, e.g., arranged in concentric rings around the imaging element 114. For example, each of the first and second arrays of light sources may include a plurality of light sources 112a, 112c spaced apart from one another in a ring around the element 114.

In the example shown, the first or outer array includes LEDs 112a configured to transmit shortwave infrared (SWIR) light, e.g., at wavelengths between about 1200-2500 nm, e.g., any of the bands described with references to the other imaging devices herein. For example, one or more of the LEDs 112a may be configured to transmit a narrow band of light centered around 1300 nm and one or more of the LEDs 112a may be configured to transmit a narrow band of light centered on a desired wavelength, e.g., about 1450 nm, 1480 nm, 1485 nm, or 2000 nm. An actuator, e.g., a switch and the like on the proximal end of the device (not shown) may be coupled to the LEDs 112a to cause the LEDs transmit one or both of these narrow bands of light, e.g., to enable switching between the two wavelengths, as desired.

In another example, an excitation source may be provided that transmits a narrow band of light maximizing the spectral absorption peak of water. For example, a 1480 nm laser diode may be used for improved water absorption compared to broadband, e.g., visible, LED light.

The second or inner array includes LEDs 112c configured to transmit infrared light below 1000 nm, e.g., between about 785-808 nm. For example, the LEDs 112c may be configured to transmit light at a narrow band centered around 785 nm. Optionally, the LEDs 112c may include a bandpass filter (not shown), e.g., to block transmission of light above a desired wavelength, e.g., above 1000 nm to prevent interference with the SWIR light sources.

Alternatively, one or both arrays of LEDs may be replaced with laser diodes or a separate light source (not shown) may be provided, e.g., connected to the proximal end of the imaging device 110 and delivering light through one or more optical fibers (also not shown), similar to other devices herein.

Optionally, as shown in FIG. 4A a polarizer 113 may be optically coupled to the light source, e.g., to polarize light transmitted by one or both arrays of LEDs 112. For example, an annular polarizer film may be mounted over the LEDs 112 to polarize the transmitted light, i.e., without covering the camera 114. Optionally, the polarizer may be movable, e.g., rotatable relative to the distal surface of the distal tip 124 to allow the polarization to be rotated in front of the camera 114.

The camera 114 may include an indium gallium arsenide (InGaAs) or other camera, e.g., including one or more focal plane arrays (FPAs) that may be used to acquire images illuminated by one or more of the wavelengths of the light source 112, similar to the other devices herein. The FPA of the camera 114 may be mounted on the distal end 124, e.g., on the distal surface, or may be mounted in an external device (not shown) that receives images via optical fibers extending from the distal end 124, similar to the other devices herein. The camera 114 may include one or more lenses and/or cameras on the distal end 1124, if desired.

For example, as shown in FIG. 4A, an optional filter holder 115 may be provided that may allow a specific filter (not shown) to be secured to the distal end 124 over the camera 114 (or the camera lens). For example, a desired long pass filter (not shown) may be inserted into the holder to filter the light received by the camera 114, as desired.

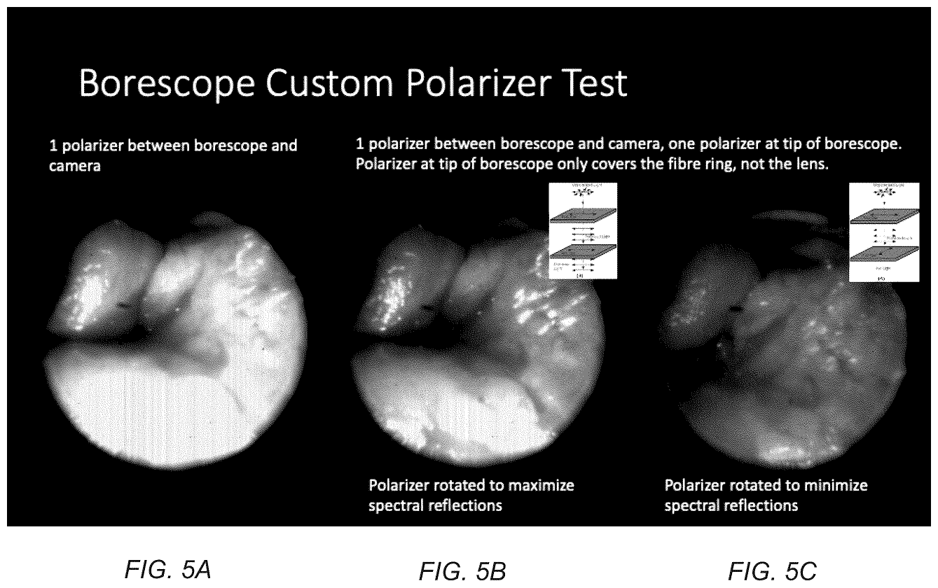

FIGS. 5A-5C show exemplary images of tissue structures imaged using the device 110. For example, FIG. 5A on the left shows an exemplary image with a polarizer positioned in the light path between the light source 112 and the camera 114. FIGS. 5B and 5B show exemplary images in which the polarizer only covers the light source 112 and rotated to maximize spectral reflections (FIG. 5B in the center) and minimize spectral reflections (FIG. 5C on the right).

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An imaging device, comprising:
an imaging device comprising a proximal end, a distal end sized for introduction into the body, and an imaging element carried by the distal end;
a light source coupled to the imaging device to selectively deliver one or both of short wavelength infrared (SWIR) light and visible light from the distal end; and
a camera for acquiring images via the imaging element.

2. The imaging device of claim 1, wherein the light source is configured to generate narrow band SWIR light to the imaging element at a wavelength between about 1200-2500 nm.

3. The imaging device of claim 1, wherein the light source is configured to deliver SWIR light from the distal end at a narrow band centered at one or more water absorption bands.

4. The imaging device of claim 1, wherein the light source is configured to deliver SWIR light from the distal end at one or more wavelengths to allow imaging through blood.

5. The imaging device of claim 1, wherein the light source is configured to generate SWIR light to the imaging element at a narrow band centered on a wavelength of about 1450 nm, 1480 nm, 1485 nm, or 2000 nm.

6. The imaging device of claim 1, wherein the light source is configured to generate SWIR light to the imaging element at a narrow band centered on a wavelength of about 1300 nm, 1450 nm, 1550 nm, or 1650 nm.

7. The imaging device of claim 1, wherein the SWIR light source comprises one or more LEDs, lasers, or halogen lights.

8. The imaging device of claim 1, further comprising:
a first polarizer optically coupled to the imaging element to polarize incident light from the light source delivered from the distal end; and
a second polarizer optically coupled to the camera to further polarize reflected light captured by the imaging element and delivered to the camera.

9. The imaging device of claim 8, wherein the first polarizer is orthogonal to the second polarizer.

10. The imaging device of claim 8, wherein one of the first and second polarizers is rotatable to adjust the amount of polarization.

11. The imaging device of claim 1, wherein the visible light source is configured to generate white light.

12. The imaging device of claim 1, further comprising one or more actuators coupled to the light source to alternatively deliver the SWIR light source and the visible light.

13. The imaging device of claim 1, further comprising a display for presenting images acquired by the camera.

14. The imaging device of claim 13, further comprising a processor coupled to the camera and the display for processing signals from the camera corresponding to the images acquired by the imaging element to present the images on the display.

15. The imaging device of claim 14, wherein the processor is configured to process the signals from the camera to superimpose SWIR light images onto visible light images on the display.

16-24. (canceled)

25. The imaging device of claim 1, further comprising a first polarizer on the distal end configured to polarizer light transmitted by the light source.

26. The imaging device of claim 25, wherein the first polarizer comprises a ring mounted over the light source to polarize the transmitted light without covering the imaging element.

27. The device of claim 25, wherein the first polarizer comprises a ring mounted over a distal end of the imaging device to polarize the transmitted light from the light source without covering a lens optically coupled to the camera.

28. The device of claim 25, further comprising:
a video coupler optically coupling the camera to the proximal end of the imaging device; and
a second polarizer within the video coupler to polarize reflected light captured by the lens configured to reduce bright reflections.

29-32. (canceled)

33. A method for medical imaging, comprising:
generating short wavelength infrared (SWIR) light at a narrow band centered at one or more water absorption bands; and
acquiring images via an imaging element.

34-56. (canceled)

57. An imaging device, comprising:
an elongate member comprising a proximal end, a distal end sized for introduction into the body, and an imaging element carried by the distal end;
a laser light source coupled to the proximal end configured to generate short wavelength infrared (SWIR) light to the imaging element at a narrow band centered at one or more of about 1450 nm, 1480 nm, 1485 nm, or 2000 nm;
a camera for acquiring images via the imaging element;
a first polarizer optically coupled to the imaging element to polarize incident light from the light source delivered from the distal end; and
a second polarizer optically coupled to the camera to further polarize reflected light captured by the imaging element and delivered to the camera.

58. (canceled)
